(19) United States
(12) Patent Application Publication
(10) Pub. No.: US 2004/0225019 A1
(43) Pub. Date: Nov. 11, 2004

(54) Methods of preventing or treating motion sickness

(76) Inventors: Jay C. Buckey, Hanover, NH (US); Larry R. Brown, Newton, MA (US); Donna L. Alvarenga, Hanover, NH (US)

Correspondence Address: Jane Massey Licata, Licata & Tyrrell P.C., 66 E. Main Street, Marlton, NJ, 08053, US

(21) Appl. No.: 10/786,429

(22) Filed: Feb. 25, 2004

Related U.S. Application Data
(60) Provisional application No. 60/450,132, filed on Feb. 25, 2003.

(51) Int. Cl. A61K 31/137

(52) U.S. Cl. 514/649

(57) ABSTRACT

The present invention provides a method of preventing or treating motion sickness by orally or topically administering a halogenated pheniramine.

This application claims the benefit of priority from U.S. patent application Ser. No. 60/450,132 filed Feb. 25, 2003, which is incorporated herein by reference in its entirety.

This invention was made in the course of research sponsored by the Office of Naval Research (ONR Grant No. N00014-00-1-0694). The U.S. government may have certain rights in this invention.

### BACKGROUND OF THE INVENTION

Motion sickness is an illness which typically occurs when humans are subjected to long-lasting external movement or transportation accompanied by unusual movements such as shaking, waving, atmospheric changes (e.g., flying in an airplane), great acceleration, and uneven road conditions. Motion sickness is not viewed as a disease but as a physiological symptom complex wherein the symptoms experienced, of which nausea and vomiting are common, depend on the individual in question. When the individual experiences motion sickness in a work environment, i.e., truck drivers, air pilots, air craft staff members and the like, the potential for a disadvantageous and dangerous condition result. Such individuals are often required to exhibit high level concentration and intellect, and the presence of motion sickness symptoms can severely detract from their ability to do so.

Motion sickness is also a problem in simulators and with virtual reality training (Regan and Price (1994) Aviat. Space Environ. Med. 65(6):527-30; Blok (1992) Milit. Med. 157(3):109-11; Kennedy, et al. (1989) Aviat. Space Environ. Med. 60(1):10-6). Sickness in this environment can have negative effects on learning, and produce a lack of confidence in the training device. Current therapies for motion sickness, including transdermal scopolamine (TransdermScop®), dymendydrinate or promethazine, are effective drugs but also sedating (Wood, et al. (1984) Aviat. Space Environ. Med. 55(2):113-6; Babe and Serafin (1996) Babe and Serafin (1996) In: Goodman and Gilman's The Pharmacologic Basis of Therapeutics. 9th ed., Hardman and Limbird (Ed.) New York, McGraw Hill, pp.581-600). The sedative side effect is undesirable if an emergency or other demanding operational task should arise which would demand full alertness.

Chlorpheniramine is one of the first generation reversible antagonists to the H₁-receptor involved in allergic responses. Pyridyl aliphatic amines with halogenated aryl groups, like chlorpheniramine, possess potent antihistaminic activity, while causing less sedation than the ethanolamine (such as diphenhydramine) and the ethylenediamine classes of antihistamines. Among a series of 70 different compounds screened in guinea pigs, chlorpheniramine possessed the greatest antihistaminic potency and therapeutic index [LD₅₀ divided by the ED₅₀] (Labelle and Tislow (1955) J. Pharma. Exp. Ther. 113:72-88) of the H1-histamine antagonists. Chlorpheniramine, sold under the common brand names CHLOR-TRIMETON® and CODIMAL-A® is typically administered orally. A chlorpheniramine cream is also available as POLARAMINE®.

Antihistamine compositions containing chlorpheniramine and penetration enhancers have been described for use in patches, creams, lotions, and gels (U.S. Patent Application No. 20020028235).

U.S. Pat. No. 3,227,157 provides a topical delivery system for chlorpheniramine as an antihistamine using enzymatic degradation of a polymer slab.

U.S. Pat. No. 5,422,118 discloses transdermal administration of alkyl amine antihistamines, such as chlorpheniramine, to maintain a high transdermal flux rate.

The dextrorotatory isomer of chlorpheniramine, dexchlorpheniramine, possesses much greater antihistaminic activity than either the levorotatory isomer or the racemic mixture (Roth and Govier (1958) J. Pharma. Exp. Ther. 124:347-349; Hill, et al. (1977) Nature 270(5635):361-3). The potency ratio of dexchlorpheniramine to racemic chlorpheniramine to levochlorpheniramine is in the order of 100:50:1 (Roth and Govier (1958) supra). The therapeutic index of dexchlorpheniramine is among the highest ever measured for an antihistamine. Thus, dexchlorpheniramine is safe and effective in relatively small doses and, although it has twice the potency of the racemic mixture, the potency is not correlated with toxicity (Goldenthal (1971) Tox. Appl. Pharma. 18(1):185-207).

Chlorpheniramine and its stereoisomers have been observed to have dose-related mild central nervous system (CNS) depression and CNS stimulation (Bergman (1990) Psychopharmacology 100(1):132-4; Sakurai, et al. (1991) Tohoku J. Exp. Med. 163(4):239-44). The CNS depression is believed to be mediated by the blockade of central histamine receptors. This blockade is stereoselective, with the d-isomer being more potent. CNS stimulation occurs at higher doses of chlorpheniramine and its stereoisomers; and may be due to a non-stereospecific blockade of sites mediating the neuronal reuptake of dopamine and serotonin. By blocking the neuronal reuptake of CNS-stimulatory neurotransmitters, chlorpheniramine and its stereoisomers might prolong the effect of these transmitters at their receptors, resulting in CNS stimulation. Chlorpheniramine is only a mild CNS depressant and may even stimulate the CNS at higher doses. Thus, on a weight basis, chlorpheniramine is at least six-times more potent than other H₁-antihistamines. The dexchlorpheniramine form possesses twice the potency of the racemic mixture and may possibly have fewer side effects because of the levorotatory form has virtually no antihistaminic activity.

H₁-receptor antagonists such doxepin, triprolidine, pheniramine, meclizine, and diphenhydramine have been shown to be useful for symptomatic treatment of motion sickness (Chinn, et al. (1952) Am. J. Med. 12:433-439; Chinn, et al. (1953) J. Pharmacol. Exp. Thera. 108:69-79; Kohl (1991) J. Respir. Dis. 12:S17-21; Wood and Graybiel (1968) Aero. Med. 39(12):1341-4; Wang and Dutia (1995) Exp. Brain Res. 105(1):18-24). The effectiveness of chlorpheniramine to decrease symptoms associated with motion sickness has not been demonstrated. It has now been shown that chlorpheniramine is useful to prevent or treat motion sickness with reduced sedation.

### SUMMARY OF THE INVENTION

The present invention provides a method for preventing or treating motion sickness. The method involves administering to a susceptible subject or a subject exhibiting signs or symptoms of motion sickness an effective amount of a halogenated pheniramine to prevent, reduce, or decrease the signs or symptoms of motion sickness. In particular embodiments, the halogenated pheniramine is chlorpheniramine, brompheniramine or enantiomers thereof. In other embodiments, the halogenated pheniramine is administered orally or topically to overcome existing signs or symptoms associated with motion sickness and to facilitate removal if side effects are experienced.

These and other aspects of the present invention are set forth in more detail in the following description of the invention.

### DETAILED DESCRIPTION OF THE INVENTION

It has now been found that a halogenated pheniramine, chlorpheniramine, lengthens the time that subjects can tolerate motion without symptoms of motion sickness. This halogenated pheniramine does not affect performance as determined by serial addition, memory, or tracking. As used herein, the term halogenated pheniramine includes, but is not limited to, chlorpheniramine and brompheniramine and includes all enantiomers, isomers, tautomers and salts of thereof including, for example, dexchlorpheniramine, dexbrompheniramine and levochlorpheniramine.

Using an off-vertical rotation chair, two doses of chlorpheniramine (low dose of 4 mg and high dose of 12 mg) were administered to subjects to analyze the effects of chlorpheniramine on motion sickness. High dose chlorpheniramine significantly increased chair time (p=0.001)(Table 1).

TABLE 1

| | Mean Time | | |
|---|---|---|---|
| Treatment | (min., n = 18) | Std. Dev. | Std. Error |
| Placebo | 7.2 | 4.4 | 1.0 |
| Low Dose | 10.2 | 5.7 | 1.4 |
| High Dose | 11.7 | 6.4 | 1.5 |

Chair time increased from a mean of 7.2 minutes with placebo to a mean of 11.7 minutes with high-dose chlorpheniramine, a 63% increase. Three subjects increased their chair time to the maximum (the chair rides were stopped at 20 minutes).

Motion sickness severity scores were also analyzed to determine if the subjects were reporting fewer or less severe symptoms of motion sickness. The calculated motion sickness severity scores at the end of the chair rides were significantly less with high dose than with the placebo (p=0.0004) and low dose (p=0.01)(Table 2).

TABLE 2

| | Mean Motion Sickness | | |
|---|---|---|---|
| Treatment | Severity Score (n = 18) | Std. Dev. | Std. Error |
| Placebo | 18.2 | 6.0 | 1.4 |
| Low Dose | 16.4 | 6.5 | 1.5 |
| High Dose | 12.4 | 7.0 | 1.6 |

Results of cognitive testing indicated that performance in the objective tests of addition, tracking and memory tasks were not significantly affected. Reaction time, however, was significantly increased (p=0.0002) after the chair rides with high dose chlorpheniramine (Table 3).

TABLE 3

| | | Mean Reaction | | |
|---|---|---|---|---|
| | | Time | | |
| Treatment | Time* | (msec, n = 15) | Std. Dev. | Std. Error |
| Placebo | PreDrug | 278.8 | 42.8 | 11.1 |
| | PostDrug | 267.4 | 31.0 | 8.0 |
| | PostRide | 292.0 | 40.9 | 10.6 |
| Low Dose | PreDrug | 271.2 | 44.1 | 11.4 |
| | PostDrug | 286.1 | 56.9 | 14.7 |
| | PostRide | 295.2 | 39.6 | 10.2 |
| High Dose | PreDrug | 265.1 | 31.0 | 8.0 |
| | PostDrug | 281.5 | 41.6 | 10.7 |
| | PostRide | 298.4 | 46.9 | 12.1 |

*Time indicates when measurements were taken relative to the drug administration and chair rides.

In the subjective tests, significant increases were obtained in the Karolinksa sleepiness scores (p<0.0001) after the chair rides. With high dose chlorpheniramine treatment, higher sleepiness scores were obtained, scores on the “alert-sleepy” mood scale significantly changed toward the sleepy portion of the scale (p<0.0001), and scores on the “clearheaded-groggy” mood scale significantly changed toward the groggy portion of the scale (p<0.0001).

Subjects were asked after each chair ride to answer whether they thought they had received placebo or active drug. They were also asked whether they had had any side effect or toxicity from the drug and to note any other symptoms that they had experienced after leaving the testing area. The most commonly reported toxicities were drowsiness and dry mouth. Seven out of 18 subjects (39%) did not think that they had received any drug even though they had taken high dose chlorpheniramine. Therefore, the subjects were more likely to correctly identify when they had received active drug when they had received chlorpheniramine. The subjects could not differentiate whether they had received high dose or low dose. While there was a tendency for the subjects to be more likely to report a side effect with chlorpheniramine, this did not reach statistical significance.

Accordingly, one aspect of the invention provides a method of preventing or treating motion sickness. The method involves administering to a susceptible subject, or a subject exhibiting signs or symptoms of motion sickness, an effective amount of a halogenated pheniramine. An effective amount of a halogenated pheniramine is an amount which when administered prevents, reduces, decreases, or eliminates the signs or symptoms associated with motion sickness including, but not limited to, nausea, drowsiness, headache, increased salivation, nausea, stomach awareness, stomach discomfort, pallor, cold sweating, clammy skin, dizziness and vomiting.

A halogenated pheniramine for prevention or treatment of motion sickness can be administered by any suitable means, including parenteral injection (such as intraperitoneal, subcutaneous, or intramuscular injection), orally, or by topical application (e.g., transdermal or to an airway surface). When administered orally, the halogenated pheniramine can be in the form of a liquid, pill, or capsule. In one embodiment, the halogenated pheniramine for prevention or treatment of motion sickness is administered topically as a cream, lotion, liquid, ointment, gel, tattoo, patch or aerosol.

In an embodiment of the present invention, topical administration of the halogenated pheniramine is to an airway surface, carried out by intranasal administration (e.g., by use of dropper, swab, or inhaler which deposits a pharmaceutical formulation intranasally). Topical application of the halogenated pheniramine to an airway surface can also be carried out by inhalation administration, such as by creating respirable particles of a pharmaceutical formulation (including both solid particles and liquid particles) containing the halogenated pheniramine as an aerosol suspension, and then causing the subject to inhale the respirable particles. Methods and apparatus for administering respirable particles of pharmaceutical formulations are well known, and any conventional technique can be employed.

In a particular embodiment of the invention, the halogenated pheniramine for prevention or treatment of motion sickness is administered transdermally via a patch. In an alternative embodiment, the halogenated pheniramine for prevention or treatment of motion sickness is administered transdermally via a temporary tattoo. Most H1-antihistamines are not suitable for transdermal administration, however a halogenated pheniramine, which is a small molecule with a relatively low effective dose level, can be effectively administered transdermally. Transdermal administration has the advantage of increasing the blood concentration of the halogenated pheniramine gradually to a relative constant concentration that results in the elimination of drug concentration peaks, which can be associated with side effects. Once a steady state concentration of the halogenated pheniramine has been achieved by diffusion through the skin, constant levels of the halogenated pheniramine can be maintained in the blood. A further advantage of transdermal administration is that oral administration is not as effective in motion sickness once sickness begins, since motion sickness slows gastric motility. Also, the oral pharmacokinetics of many drugs in space is not well established and can differ from ground-based measurements (Tietze and Putcha (1994) J. Clin. Pharmacol. 34(6):671-6). Moreover, if side effects are experienced, the drug can be removed. With intramuscular or oral administration, an individual experiencing sedation or other side effects must wait for the drug to be metabolized.

Plasma drug concentrations delivered transdermally can be regulated by varying the size of patch or tattoo applied to the subject to keep drug concentrations within the therapeutic range and avoid side effects associated with bolus drug administration. Patches and tattoos and their preparation for use in the administration of a halogenated pheniramine according to the method of the invention are provided, for example, in U.S. Patent Application No. 20020187181, U.S. Pat. Nos. 6,315,480 and 6,267,983, herein incorporated by reference in their entirety.

Topical formulations of the halogenated pheniramine can be prepared according to known methods of producing pharmaceutical formulations, whereby the halogenated pheniramine is combined in admixture with a pharmaceutically acceptable carrier. Pharmaceutically acceptable carriers are provided, for example, in Remington's Pharmaceutical Sciences (16th ed., Osol, A. ed., Mack Easton Pa. (1980)). In order to form a halogenated pheniramine composition suitable for administration, such compositions will contain an effective amount of the halogenated pheniramine together with a suitable amount of a carrier, excipient, or stabilizer which is nontoxic to the cell or mammal being exposed thereto at the dosages and concentrations employed. Often the carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Halogenated pheniramine compositions can be administered alone or in combination with other agents which prevent or treat signs or symptoms associated with motion sickness including, but not limited to, buspirone, 8-OH-DPAT, scopolamine, phenyloin and dexamethasone. Moreover, the halogenated pheniramine can be administered in combination with other agents which reduce or prevent side effects which can be associated with the halogenated pheniramine, e.g., ephedrine, caffeine, dextroamphetamine, ritalin, pemoline, or modafinil to reduce drowsiness.

The exact dosage will be dependent on factors related to the subject that requires treatment. Dosage can be adjusted to provide sufficient levels of the halogenated pheniramine or to maintain the desired effect of preventing or reducing the severity of the motion sickness. Factors which can be taken into account include the severity of the existing symptoms, general health of the subject, age, weight, and gender of the subject and general susceptibility to motion sickness. Typical ranges of the halogenated pheniramine which can be administered to effectively prevent or treat the signs or symptoms associated with motion sickness are 1 to 25 mg, or 4 to 12.

When used in combination with other agents, the dosage of the other agent used will be dependent on the agent and the formulation thereof. To illustrate, a 12 mg dose of oral chlorpheniramine may be co-administered (e.g., before, with or after) a 25 or 50 mg dose of oral ephedrine.

The invention is described in greater detail by the following non-limiting examples.

### EXAMPLE 1

### Motion Sickness Stimulus

Motion sickness was simulated by rotation in a tilted chair, also known as off-vertical rotation (Knox, et al. (1994) Laryngoscope 104(8 Pt 1):935-9; Woodard, et al. (1993) Aviat. Space Environ. Med. 64(5):363-6; Mowrey and Clayson (1982) Lancet 1(8273):655-7; Graybiel and Miller (1970) Aero. Med. 41(4):407-10). Off-vertical rotation produces motion sickness symptoms in more than 90% of subjects tested and has a high test-retest reliability. No provocative head motions are required which minimizes subject fatigue and training. For each run, the chair was tilted to 15 degrees off vertical, and accelerated at 5 degrees/sec² to 17.5 revolutions per minute. These settings are the most effective in producing motion sickness with off-axis rotation (Miller and Graybiel (1970) Aero. Med. 41(4):407-10). The subject was restrained with a seat belt and shoulder harness. The subject's head was also secured to the headrest with a strap to prevent head movements. Opaque goggles were placed over the subject's eyes.

### EXAMPLE 2

### Motion Sickness Assessment

The degree of motion sickness was assessed in three different ways. For the first method, the subjects were instructed how to use a well-known ratio scaling method (Bock and Oman (1982) Aviat. Space Environ. Med. 53(8):773-7) and were given a familiarization run in the chair to experience the motion sickness symptoms. The subjects were instructed to rate the peak overall discomfort they experienced in the familiarization ride as a “10” and to remember this level. They were instructed to rate all other levels of discomfort relative to this level. For example, discomfort which was half as severe would be rated as a “5”. This method is similar other well-known methods (see, e.g., Stott, et al. (1989) Br. J. Clin. Pharma. 27(2):147-57).

The second method was symptom recording. Prior to the familiarization run the subjects were instructed about the different symptoms that might occur (drowsiness, epigastric awareness, and the like). The subjects were asked about these symptoms each minute during the rotation and logged on a scoring sheet (Miller and Graybiel (1970) Aero. Med. 41(4):407-10), which was a variant of the Pensacola Diagnostic Rating Scale. The symptoms were assigned points and from these points, five levels of severity were determined (frank sickness, severe malaise, moderate malaise A, moderate malaise B, slight malaise).

The third method used to assess motion sickness was to record the total number of minutes the subject rides in the chair before stopping (Woodard, et al. (1993) supra; Mowrey and Clayson (1982) supra).

### EXAMPLE 3

### Subject Characteristics

Subjects did not have any active illnesses and were not taking any medications. They had no history of vertigo, Meniere's disease, labyrinthine dysfunction or other neuro-otological disease. Urine pregnancy tests were self administered by the female subjects to rule out pregnancy prior to study drug ingestion. The subjects were screened for allergies and for antihistamine use. Each subject had a motion sickness susceptibility quotient measured using standard methods (Reason and Brand (1975) In: Motion Sickness. London, Academic Press). All subjects had a normal audiogram and normal examination of neurological function. Nine men and nine women participated. The subject characteristics are provided in Table 4.

TABLE 4

| | Age | Height | Weight | | |
|---|---|---|---|---|---|
| | (Years) | (cm) | (kg) | BSA | MSQ |
| Mean | 30 | 173 | 70.6 | 23.6 | 38.7 |
| S.D. | 8.4 | 9.8 | 11.3 | 8.4 | 28.8 |

BSA = body surface area,
MSQ = motion sickness quotient.

### EXAMPLE 4

### Study Design

The studies employed a double-blind, placebo controlled, crossover design. Two doses of chlorpheniramine were analyzed; a low dose of 4 mg and a high dose of 12 mg. The placebo was a single lactose capsule. Chlorpheniramine and placebo capsules looked identical. The medications were labeled only with the subject's name, identification number and sequence of administration (i.e., Run 1, Run 2, Run 3). To avoid ordering effects, subjects were randomly assigned to one of six possible administration sequences of placebo, low dose chlorpheniramine and high dose chlorpheniramine. Both the research team and subjects were unaware of the order of administration.

At least one week before beginning the experimental study and after giving informed consent, each subject had a baseline measurement of cognitive function and a familiarization ride in the chair. Prior to the ride, subjects received a briefing on the symptoms they might experience, how to report those symptoms and instructions on when to stop the test. After the familiarization ride the subjects were instructed to remember their peak overall discomfort as a “10” and to rate all future symptoms relative to that baseline. All 18 subjects in the chlorpheniramine study were susceptible to the rotating chair.

The subjects rode in the chair once for each treatment (low dose, high dose, and placebo) with at least one week between rides. A one week interval between rides is sufficient to prevent habituation to the rotating stimulus (Kohl (1987) Aviat. Space Environ. Med. 58(2):125-31; Stott, et al (1989) supra).

Test medications were taken two hours after finishing breakfast. Subjects did not consume alcohol or take any medications (prescription or over the counter) for 24 hours prior to the study. On the study day, the subjects came to the lab, performed the cognitive testing battery and were observed taking the test medication. The time of drug ingestion was documented and the subjects returned three hours after taking the medication to complete the cognitive testing battery and ride in the chair. Prior to entering the chair, subjects received a standardized test briefing. Every minute during the testing, subjects were quizzed for motion sickness symptoms and asked to rate their own symptoms on a ratio scale. Testing continued until either 20 minutes elapsed, the subject requested testing stop, or severe nausea or vomiting was experienced (i.e., the subject reached a subjective 10 on their scale). Blood pressure, respiratory rate and pulse were measured before and after the chair ride using a Critikon DINAMAP® 1846-SX device.

After the chair run subjects completed a questionnaire about side effects and then again performed the cognitive testing battery.

### EXAMPLE 5

### Cognitive Function Assessment

A standard cognitive assessment battery (Dinges, et al. (1997) Sleep 20(4):267-7; Neri, et al. (1995) Aviat. Space Environ. Med 66(4):313-9; Richardson, et al. (1996) Sleep 19(9):718-26) used in studies of sleep deprivation (Jewett, et al. (1999) Sleep 22(2):171-9) was used which included both objective and subjective measures of performance and wakefulness. The components of the testing battery are provided in Table 5. The overall battery takes 20 minutes to administer and has been used successfully on the Neurolab space shuttle mission to assess how sleep loss affected crewmembers.

TABLE 5

| Test | Class | Description |
|---|---|---|
| Psychomotor | Objective | Measures changes in attention. |
| Vigilance | | A counter appears and the |
| | | subject must respond as soon as |
| | | the numbers begin counting. |
| Addition | Objective | Sequentially perform as many |
| | | additions of two-digit numbers |
| | | as possible in a fixed period |
| | | of time. |
| Tracking | Objective | Maintain a cursor in the center |
| | | of the screen using a |
| | | trackball. |
| Memory | Objective | Recall six unrelated work pairs |
| | | after 10 minutes. |
| Mood | Subjective | Visual analog scales labeled |
| | | with adjectives (i.e., alert, |
| | | competent). Subject responds |
| | | along a continuum. |
| Effort | Subjective | Subject provides self- |
| | | assessment on how well they |
| | | did, how difficult it was and |
| | | whether they could have done |
| | | better. |
| Sleepiness | Subjective | Karolinska sleepiness scale. |
| | | Subject provides a rating of |
| | | subjective sleepiness and |
| | | fatigue. |

The cognitive tests were performed three times for each chair ride. The first test served as a baseline, the second test was administered at the peak of drug effect and the last test was after the chair ride. In this design, the results of the second test reflect changes due to drug effect, while the results from the third test reflect a combination of drug effect and motion sickness effects.

### EXAMPLE 6

### Statistical Analysis

For the chair rides, the primary statistical analysis was a repeated measures, two-way, analysis for variance (ANOVA) with a Bonferroni correction for multiple comparisons. The primary outcome variable for this comparison was the duration of the chair run. The motion sickness severity scores during the runs were also analyzed in this way. Analysis of the subscales on the cognitive testing battery was also performed using a two-way, repeated measures ANOVA with a Bonferroni correction for multiple comparisons. Computer failures during the cognitive testing runs led to three incomplete data sets. The answers to questions on side effects where analyzed with a chi-square analysis.

What is claimed is:

1. A method for preventing or treating motion sickness comprising administering to a subject an effective amount of a halogenated pheniramine to decrease the signs or symptoms of motion sickness.

2. The method of claim 1, wherein the halogenated pheniramine comprises chlorpheniramine, brompheniramine or enantiomers thereof.

3. The method of claim 1, wherein the halogenated pheniramine is administered orally or topically.
